(19) United States
(12) Patent Application Publication
(10) Pub. No.: US 2017/0035615 A1
(43) Pub. Date: Feb. 9, 2017

(54) WELDING MASK

(71) Applicant: Ningbo Geostar Photoelectric Technology Co., Ltd, Ningbo (CN)

(72) Inventor: Zhaolin YANG, Ningbo (CN)

(21) Appl. No.: 15/017,939

(22) Filed: Feb. 8, 2016

(30) Foreign Application Priority Data
Aug. 6, 2015 (CN) 201520587422.3

(51) Int. Cl. A61F 9/06

(52) U.S. Cl. CPC A61F 9/06

(57) ABSTRACT

The present patent application discloses a weld mask, which has a weld cap, a protection plate, a filter and a press block. The weld cap has a through-hole. A scaleboard pedestal is installed on the edge of the through-hole. The scaleboard pedestal raises towards the inside of the weld cap and encompasses the through-hole. The protection plate, the filter and the press block are installed in the scaleboard pedestal successively. A raised snap-gauge is installed on the outside of the scaleboard pedestal. The snap-gauge includes a hole. An elastic snap joint is installed on the edge of the press block. The snap joint includes a hook and a reducible resilient trip. The top and bottom of the resilient trip connect with the press block and the hook respectively.

### CLAIM OF PRIORITY

This application claims priority to Chinese Application No. 201520587422.3 filed Aug. 6, 2015, the contents of which are hereby incorporated by reference herein.

### FIELD OF THE EMBODIMENTS

The workers use a safeguard weld cap when they weld to protect their eyes and heads. The traditional safeguard weld cap generally includes a filter and a protection plate to protect the eyes of the user. However, the filter and the protection plate are generally fixed by multiple bolts. So, the disassembly and assembly of the weld mask is very slow and troublesome.

### BACKGROUND OF THE EMBODIMENTS

The workers use a safeguard weld cap when they weld to protect their eyes and heads. The traditional safeguard weld cap generally includes a filter and a protection plate to protect the eyes of the user. However, the filter and the protection plate are generally fixed by multiple bolts. So, the disassembly and assembly of the weld mask is very slow and troublesome.

Various apparatus and devices are known in the art. However, their structure and means of operation are substantially different from the present invention. At least one embodiment of this invention is presented in the drawings below, and will be described in more detail herein.

### SUMMARY OF THE EMBODIMENTS

The present patent application aims to make the filter and the protection plate can be assembled into the weld cap more simply.

In order to solve the problems above, the present patent application provides a weld mask which comprises a weld cap, a protection plate, a filter and a press block. The weld cap comprises a through-hole. A scaleboard pedestal is installed on the edge of the through-hole. The scaleboard pedestal raises towards the inside of the weld cap and encompasses the through-hole. The protection plate, the filter and the press block are installed in the scaleboard pedestal successively. A raised snap-gauge is installed on the outside of the scaleboard pedestal. The snap-gauge includes a hole. An elastic snap joint is installed on the edge of the press block. The snap joint includes a hook and a reducible resilient strip. The top and bottom of the resilient strip connect with the press block and the hook respectively. The hook threads the hole and connects with the top of the hole so that the protection plate and the filter are fasten to the weld cap by the press block. The weld mask of the present patent application can be disassembled and assembled quickly, and the operation is very convenient.

In the weld mask of the present patent application, the snap joint comprises a button, the button is installed on the resilient strip and drives the hook and the resilient strip to press down.

In the weld mask of the present patent application, the button and the resilient strip form a trepanning, the trepanning connect with the snap-gauge, the hook is installed in the trepanning.

In the weld mask of the present patent application, some stiffener is installed between the snap-gauge and the weld cap, the press block and the weld cap, the resilient strip and the press block, the hook and the resilient strip.

In the weld mask of the present patent application, when the resilient strip is pressed down, the hook and the button can reset.

In the weld mask of the present patent application, the top of the press block connects with the top of the scaleboard pedestal and can rotate, the snap joint and the snap-gauge are installed respectively on the underneath of the press block and the scaleboard pedestal.

In the weld mask of the present patent application, by setting snap joint with a button and snap-gauge, the disassembly and assembly between the protection plate, the filter and the weld cap are much more convenient and quick.

### BRIEF DESCRIPTION OF THE DRAWINGS

FIG. 1 is the first perspective view of the weld mask, according to an embodiment of the present patent application.

FIG. 2 is the second perspective view of the weld mask, according to an embodiment of the present patent application.

FIG. 3 is the exploded view of the weld mask.

FIG. 4 illustrates the weld mask being used.

FIG. 5 is the first perspective view of the press block.

FIG. 6 is the second perspective view of the press block.

FIG. 7 is the enlarged view of A in FIG. 2.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

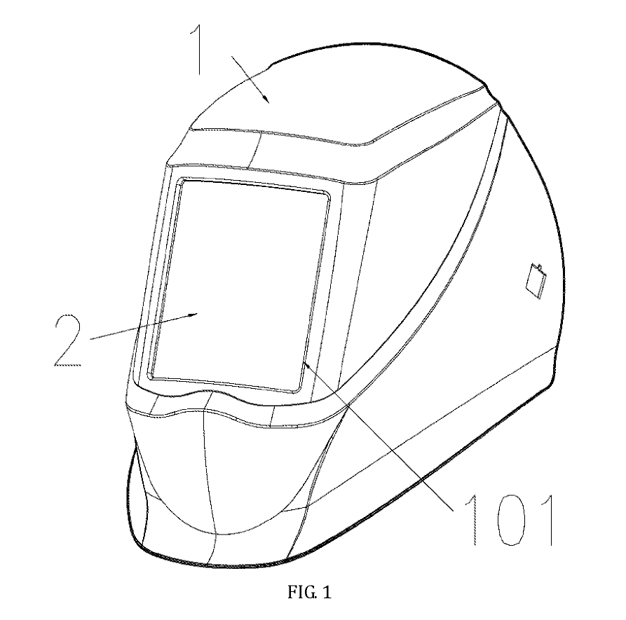

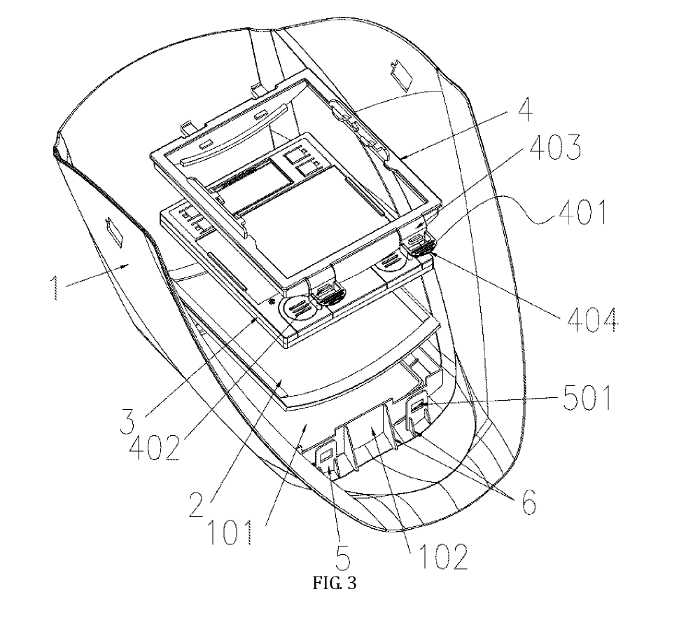

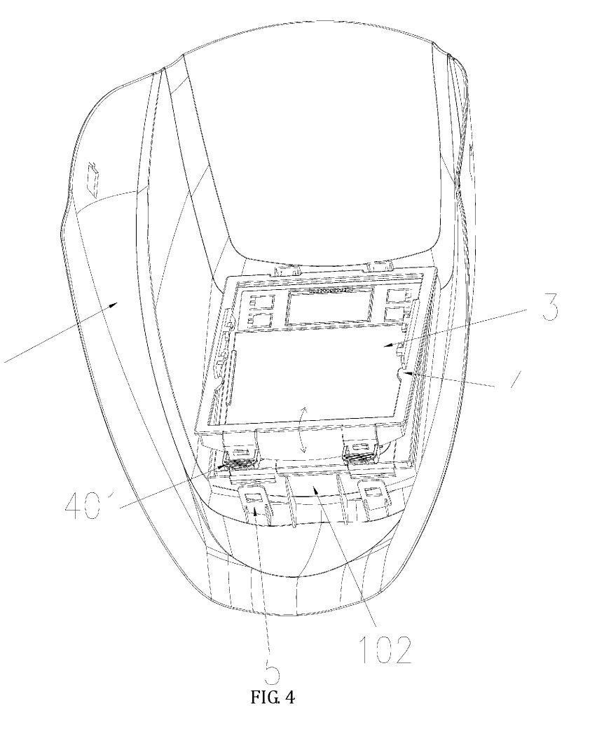

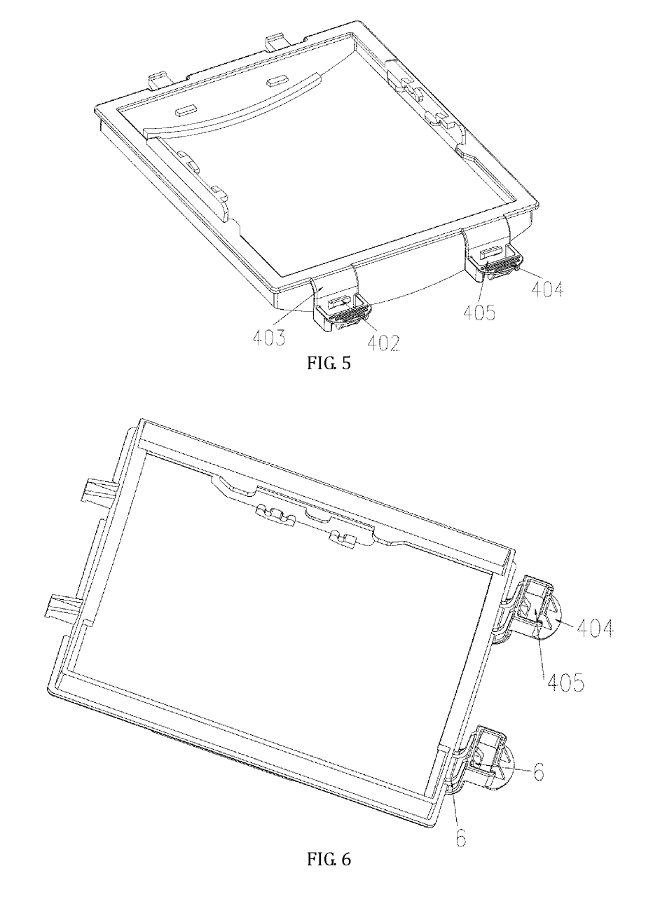

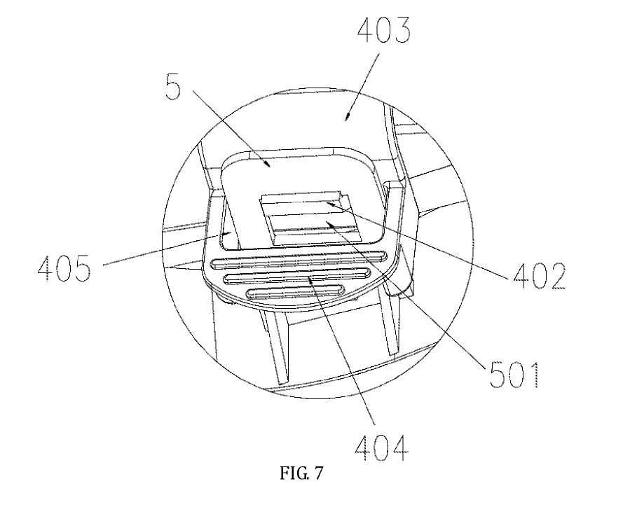

The principles of the weld mask in the present patent application will be further described with reference to the embodiments and the drawings. FIG. 1 through FIG. 7 contain reference numerals which may be explained as follows: 1: weld cap, 2: protection plate, 3: filter, 4: press block, 5: snap-gauge, 6: stiffener, 101: through-hole, 102: scaleboard pedestal, 103: bayonet, 501: hole, 401: snap joint, 402: hook, 403: resilient strip, 404: button, and 405: trepanning.

As shown in FIG. 1 to FIG. 7, a weld mask includes a weld cap 1, a protection plate 2, a filter 3 and a press block 4. The weld cap 1 comprises a through-hole 101. A scaleboard pedestal 102 is installed on the edge of the through-hole 101. The scaleboard pedestal 102 raises towards the inside of the weld cap 1 and encompasses the through-hole 101. The protection plate 2, the filter 3 and the press block 4 are installed in the scaleboard pedestal 102 successively. The top of the press block 4 connects with the top of the scaleboard pedestal 102 and can rotate. A raised snap-gauge 5 is installed on the downside of the scaleboard pedestal 102 in the weld cap 1. The snap-gauge 5 includes a hole 501. An elastic snap joint 401 is installed on the downside of the press block 4. The snap joint 401 includes a hook 402, a button 404 which can be pressed and a reducible resilient strip 403. The top of the resilient strip 403 connects with the edge of the press block 4. The bottom of the resilient strip 403 connects with the button 404 and the hook 402. The button 404 and the resilient strip 403 form a trepanning 405. The trepanning 405 connects with the snap-gauge 5. The hook 402 is installed in the trepanning 405. The hook 402 threads the hole 501 and connects with the top of the hole 501.

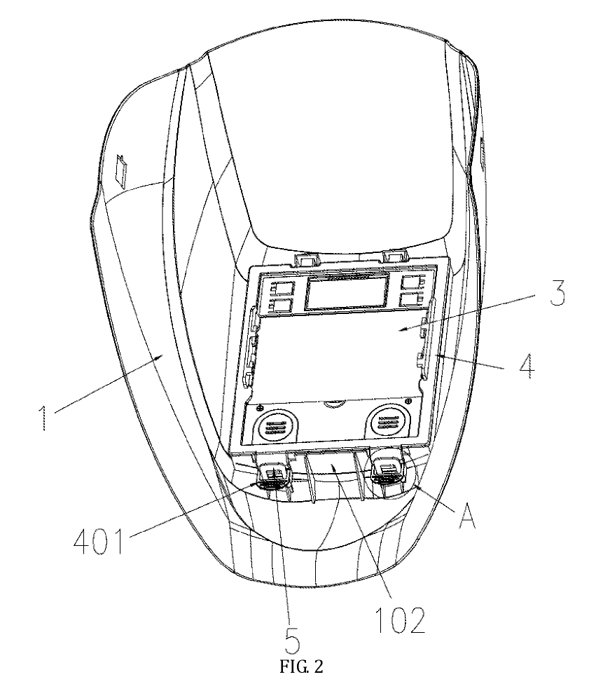

As shown in FIG. 2 to FIG. 4 and FIG. 7, when assembling, the protection plate 2, the filter 3 and the press block 4 are put in the scaleboard pedestal 102 successively. The top of the press block 4 hinges with the scaleboard pedestal 102. The snap joint 401 rotates and moves to the inner side of the weld cap 1 via the above hinge joint. The trepanning 405 just fits into the snap-gauge 5. The hook 402 moves down and when the hook 402 comes into the hole 501. By the restoration of the resilient strip 403, the hook 402 rotates and connects with the top edge of the hole 501. The protection plate 2 and the filter 3 are fasten to the weld cap 1.

As shown in FIG. 2 to FIG. 4 and FIG. 7, when disassembling, the button 404 is first pressed down. The button 404 drives the hook 402 and the resilient strip 403 to move down and rotate toward the direction of the scaleboard pedestal 102. Then the snap-gauge 5 is moved toward the scaleboard pedestal 102. The hook 402 separates from the top edge of the hole 501 and disconnects from the hole 501. Then the whole press block 4 is rotated to separate from the snap-gauge. Then the protection plate 2, the filter 3 and the weld cap 1 are separated. The resilient strip 403 drives the hook 402 and the button 404 to reset.

The disassembly and assembly is fast and convenient.

A stiffener6 is installed between the snap-gauge 5 and the weld cap 1, the press block 4 and the weld cap 1, the resilient strip 403 and the press block 4, the hook 402 and the resilient strip 403. The connective structure is more firm and doesn't rupture easily.

Of course, in the above embodiment, the top of the press block 4 and the scaleboard pedestal 102 can be not designed to be a connecting rotatable structure. It can be designed to be other connecting structures. It can use the connecting structure between the snap-gauge 5 and the snap joint 401. Or, the snap-gauge 5 and the snap joint 401 are not designed on the bottom of the press block 4 and the scaleboard pedestal 102, but on the left and right sides of the press block 4 and the scaleboard pedestal 102, thus form a symmetry connection. The disassembly and assembly is still fast and convenient than current technology.

Although the present patent application has been described with respect to certain embodiments, the description is not regarded as limiting of the present patent application. The alternative modifications, changes or improvements of aspects of the embodiments of the invention fall within the spirit of the present patent application.

While this disclosure refers to exemplary embodiments, it will be understood by those skilled in the art that various changes may be made and equivalents may be substituted for elements thereof without departing from the scope of the disclosure. In addition, many modifications will be appreciated by those skilled in the art to adapt a particular instrument, situation or material to the teachings of the disclosure without departing from the spirit thereof. Therefore, it is intended that the disclosure not be limited to the particular embodiments disclosed.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Therefore, it is intended that the disclosure not be limited to the particular embodiments disclosed.

What is claimed is:

1. A weld mask, comprising:
a weld cap (1);
a protection plate (2);
a filter (3); and
a press block (4);
wherein the weld cap (1) comprises a through-hole (101), a scaleboard pedestal(102) is installed on an edge of the through-hole (101), the scaleboard pedestal raises towards the inside of the weld cap (1) and comprises the through-hole (101), the protection plate (2), the filter (3) and the press block (4) are installed in the scaleboard pedestal (102) successively, wherein a raised snap-gauge (5) is installed on an outside of the scaleboard pedestal (102) in the weld cap (1), the snap-gauge (5) comprises a hole (501); an elastic snap joint (401) is installed on the edge of the press block (4), the snap joint (401) includes a hook (402) and a reducible resilient strip (403), a top and bottom of the resilient strip (403) connect with the press block (4) and the hook (402) respectively, the hook (402) threads the hole (501) and connects with the top of the hole (501); and the protection plate (2) and the filter (3) are fasten to the weld cap (1) by the press block (4); wherein the snap joint (401) comprises a button (404), and the button (404) is installed on the resilient strip (403) and drives the hook (402) and the resilient strip (403) to press down; wherein the button (404) and the resilient strip (403) form a trepanning (405), the trepanning (405) connects with the snap-gauge (5), and the hook (402) is installed in the trepanning (405); wherein a stiffener (6) is installed between the snap-gauge (5) and the weld cap (1), between the press block(4) and the weld cap(1), between the resilient strip (403) and the press block (4), between the hook (402) and the resilient strip (403) respectively.

2. The weld mask of claim 1 wherein when the resilient strip (403) is pressed down, the hook (402) and the button (404) resets.

3. The weld mask of claim 2, wherein a top of the press block (4) connects with a top of the scaleboard pedestal (102), the snap joint (401) and the snap-gauge (5) are installed respectively on an underneath of the press block (4) and the scaleboard pedestal (102).

4. A weld mask, comprising:
a weld cap (1);
a protection plate (2);
a filter (3); and
a press block (4);
wherein the weld cap (1) comprises a through-hole (101), a scaleboard pedestal(102) is installed on an edge of the through-hole (101), the scaleboard pedestal raises towards the inside of the weld cap (1) and comprises the through-hole (101), the protection plate (2), the filter (3) and the press block (4) are installed in the scaleboard pedestal (102) successively, wherein a raised snap-gauge (5) is installed on an outside of the scaleboard pedestal (102) in the weld cap (1), the snap-gauge (5) comprises a hole (501); an elastic snap joint (401) is installed on the edge of the press block (4), the snap joint (401) includes a hook (402) and a reducible resilient strip (403), a top and bottom of the resilient strip (403) connect with the press block (4) and the hook (402) respectively, the hook (402) threads the hole (501) and connects with the top of the hole (501); and the protection plate (2) and the filter (3) are fasten to the weld cap (1) by the press block (4).
wherein a top of the press block (4) connects with a top of the scaleboard pedestal (102), the snap joint (401) and the snap-gauge (5) are installed respectively on an underneath of the press block (4) and the scaleboard pedestal (102).

5. A weld mask, comprising:
a weld cap (1);
a protection plate (2);
a filter (3); and
a press block (4);
wherein the weld cap (1) comprises a through-hole (101), a scaleboard pedestal(102) is installed on an edge of the through-hole (101), the scaleboard pedestal raises towards the inside of the weld cap (1) and comprises the through-hole (101), the protection plate (2), the filter (3) and the press block (4) are installed in the scaleboard pedestal (102) successively, wherein a raised snap-gauge (5) is installed on an outside of the scaleboard pedestal (102) in the weld cap (1), the snap-gauge (5) comprises a hole (501); an elastic snap joint (401) is installed on the edge of the press block (4), the snap joint (401) includes a hook (402) and a reducible resilient strip (403), a top and bottom of the resilient strip (403) connect with the press block (4) and the hook (402) respectively, the hook (402) threads the hole (501) and connects with the top of the hole (501); and the protection plate (2) and the filter (3) are fasten to the weld cap (1) by the press block (4).

6. The weld mask of claim 5, wherein the snap joint (401) comprises a button (404), and the button (404) is installed on the resilient strip (403) and drives the hook (402) and the resilient strip (403) to press down.

7. The weld mask of claim 6, wherein the button (404) and the resilient strip (403) form a trepanning (405), the trepanning (405) connects with the snap-gauge (5), and the hook (402) is installed in the trepanning (405).

8. The weld mask of claim 7, wherein a stiffener (6) is installed between the snap-gauge (5) and the weld cap (1), between the press block(4) and the weld cap(1), between the resilient strip (403) and the press block (4), between the hook (402) and the resilient strip (403) respectively.

9. The weld mask of claim 6, wherein when the resilient strip (403) is pressed down, the hook (402) and the button (404) resets.

10. The weld mask of claim 5, wherein a top of the press block (4) connects with a top of the scaleboard pedestal (102), the snap joint (401) and the snap-gauge (5) are installed respectively on an underneath of the press block (4) and the scaleboard pedestal (102).
